(19) United States
(12) Patent Application Publication
(10) Pub. No.: US 2024/0325133 A1
(43) Pub. Date: Oct. 3, 2024

(54) DEVICE FOR CONTAINING ANIMALS DURING IRRADIATION BY A PROTON BEAM

(71) Applicant: UNIVERSITE DE NAMUR, Namur (BE)

(72) Inventors: Stéphane LUCAS, Namur (BE); Sébastien PENNINCKX, Namur (BE)

(21) Appl. No.: 18/576,157

(22) PCT Filed: Jun. 13, 2022
(86) PCT No.: PCT/EP2022/065951

§ 371 Date: Jan. 3, 2024

(30) Foreign Application Priority Data
Jul. 5, 2021 (EP) 21183704.2

(51) Int. Cl. A61D 99/00; A61D 7/04

(52) U.S. Cl. CPC A61D 99/00; A61D 7/04

(57) ABSTRACT

The present invention relates to a device (1) for containing animals and keeping them asleep during their irradiation by a proton beam. The animals are located in compartments (21) and kept asleep with an anesthetic gas provided to them by a dispenser (3). The device (1) is sealed, which prevents escape from the animals and leakage of anesthetic gas.

### FIELD OF THE INVENTION

The present invention relates to a device for containing animals during irradiation by a proton beam.

### BACKGROUND OF THE INVENTION

A proton therapy installation can be used for medical treatment on patients and for research and development on animals. These two applications have antagonist requirements on the room where the proton beam arrives. The medical treatment on patients require a room with a very high level of hygiene, while the research and development on animals requires to irradiate living animals, which may be detrimental for hygiene.

Moreover, because of this use of the proton therapy installation for these two applications, the time allocated for the research and development is limited. This is problematic since installing an animal takes time, and research and development on proton therapy requires to irradiate several animals to check the reproducibility of the experimental results.

### SUMMARY OF THE INVENTION

An object of the present invention is to provide a device for containing animals during irradiation by a proton beam which can be used in a room used for medical treatment on patients and which allows a high number of experiments per unit of time.

In order to fulfill this object, the invention provides for a device for containing animals during irradiation by a proton beam, comprising:
an enclosure delimiting, at least partially, several compartments, each compartment comprising an interior suitable for receiving an animal; and
a dispenser configured for dispensing an anesthetic gas into the compartments,
   wherein the device is sealed and configured in such a way that the interior of each compartment is reachable by the proton beam.

In the device according to the invention, animals are maintained asleep. This sleep helps in the precision of the proton beam irradiation since the animals do not move, and avoids an escape from animals from the containing device. Moreover, since the device is sealed, the animals are not in contact with anything in the room and there is no risk of leakage of anesthetic gas or of animal fluids. In addition, with several compartments, several animals can be handled in parallel, which allows a high number of experiments per unit of time.

Preferably, each compartment is suitable for receiving only one animal. Since the interiors of the compartments are reachable by the proton beam, there is a way to emit the proton beam such that at least part of it can arrive inside each compartment, for example through a window in a wall of the enclosure made of material letting pass the proton beam.

Animals are preferably mammals. Animals can be mice or rats for example.

The proton beam arriving on the device is preferably in the range 1 MeV-10 GeV. The proton beam may be controlled in such a way that it scans the compartments sequentially or that it irradiates the compartments at the same time.

The anesthetic gas may be isoflurane.

In an embodiment of the invention, the enclosure comprises a base plate configured to support the animals and to close a lower side of the enclosure, and a lateral enclosure system configured to create a partition between the compartments and to close the enclosure laterally. The lateral enclosure system may be movable with respect to the base plate. The lateral enclosure system is preferably at least partially in polymethyl methacrylate (Plexiglas). The base plate is preferably in a thermally conductive material like a metal, for example in aluminum. The enclosure may comprise a gasket between the base plate and the lateral enclosure system.

In an embodiment of the invention, the base plate comprises a hollow for each of the compartments, each hollow being configured to support an animal and to collects fluids and outputs released by the supported animal. Fluids and outputs can be blood, saliva, excrements, fur or anything released by the animal.

In an embodiment of the invention, the device comprises a heating arrangement in thermal contact with the supported animals through the base plate. The heating prevents hypothermia of the animals. The heating by contact via the base plate prevents dissemination of germs, which would occur if the heating was performed by air pulsing for example. The heating arrangement is preferably located, at least partially, inside the base plate.

The enclosure comprises a cover system. It is preferably configured to close a higher side of the enclosure. The cover system is placed on the lateral enclosure system. It may be movable with respect to the lateral enclosure system.

The cover system comprises a decelerating arrangement configured to decrease the energy of the proton beam. The decrease in the energy of the proton beam enables the use of a clinical proton beam for preclinical small animal irradiations in a clinical room. In other words, it is such that the proton beam becomes compatible with animal requirements. The decelerating arrangement may be suitable for decreasing the energy of the proton beam from above 100 MeV to below 50 MeV. Thus, the proton therapy installation that is made for humans treatment (with a 230 MeV proton beam) can also be used for animals irradiation (with a proton beam below 50 MeV). Moreover, the energy can be adapted according to the depth of the tissue to irradiate. For example, a thickness of 77 mm of PMMA for the decelerating arrangement makes possible to irradiate a 5-mm tumor on the surface of the animal, and a thickness of 67 mm of PMMA for the decelerating arrangement makes possible to irradiate a 5-mm tumor at a depth of 1 cm in the animal. Other embodiments of the decelerating arrangement are possible without departing from the scope of the invention. The decelerating arrangement may comprise at least one decelerating element for each compartment.

The decelerating arrangement may be called “range shifter”. The proton beam as emitted by the cyclotron may have an energy of 230 MeV. A device at the outlet of the cyclotron may decrease the energy to 100 MeV, which is thus the energy of the proton beam arriving on the device. The decelerating arrangement may decrease the energy to a range between 1 and 50 MeV, preferably between 20 and 30 MeV.

In an embodiment of the invention, the decelerating arrangement comprises several decelerating elements of various thicknesses, configured in such a way that at least two of the compartments are covered by decelerating elements of different thicknesses. Therefore, several depths within the animals can be observed on the same device. This increases the number of experiments that can be performed within a given time slot.

In an embodiment of the invention, the cover system comprises a shielding arrangement blocking the proton beam except in several holes. Only the part of the proton beam that goes through the holes arrive in the interior of the compartments. Therefore, the proton beam touches the animal only locally, and not its full body. The shielding arrangement is preferably located between the decelerating arrangement and the animals. The shielding arrangement may be made of several shielding elements, with one shielding element for each compartment. A shielding element may be made of polyethylene with for example a thickness of 2 cm or of tantalum with a thickness of 3 mm.

In an embodiment of the invention, the device comprises proton beam collimators in the holes. The collimators are preferably telescopic collimator. They can be used to avoid beam scattering and direct the proton beam to the tissue to irradiate.

In an embodiment of the invention, the holes are in a movable arrangement of the shielding arrangement, the movable arrangement being movable with respect to the dispenser in such a way that a motion of the movable arrangement moves the holes with respect to the animals. Since the dispenser is fixed with respect to the animals, the movable arrangement is thus movable with respect to the animals. The motion of the holes modifies the position of the proton beam in the interior of the compartments and the irradiated part of the animals.

In an embodiment of the invention, the holes are in movable elements of the movable arrangement, the movable elements being rotatable around an axis parallel to the holes. The direction of the holes is the direction of the proton beam. Therefore, the movable elements are rotatable around an axis parallel to the proton beam.

In an embodiment of the invention, the dispenser comprises a gas inlet and several gas outlets, each gas outlet opening into one of the compartments.

In an embodiment of the invention, the gas outlets are in a lateral wall of the dispenser, preferably in recesses of the lateral wall of the dispenser. The gas outlets can thus be close to the mouse and/or nose of the animal. They are preferably at a height of at least 5 mm from the base plate. The lateral wall surrounds the dispenser. It can be made of several portions angled with respect to each other.

In an embodiment of the invention, the device comprises a suction system for the anesthetic gas, the suction system comprising several suction inlets and a suction outlet, each suction inlet opening into one of the compartments. Each suction inlet is preferably located at less than 2 cm from the gas outlet of the compartment. The suction outlet is connected to a filter recycling the anesthetic gas.

The invention also provides for an equipment comprising, in addition to a device, a transport arrangement configured to move the device. The transport arrangement can be for example a handle attached to the device to lift the device, a tray supporting the device, or a trolley supporting the device. The equipment is preferably movable by a human being.

### BRIEF DESCRIPTION OF THE FIGURES

For a better understanding of the present invention, reference will now be made, by way of example, to the accompanying drawings in which:

FIG. 1 illustrates several elements of a device for containing animals;

FIG. 2a illustrates the upper side of a base plate;

FIG. 2b illustrates the lower side of a base plate;

FIG. 3 is a front side of several elements of a device for containing animals;

FIG. 4 is a back side of a device for containing animals; and

FIG. 5 is a cross-section in a dispenser.

### DESCRIPTION OF THE INVENTION

The present invention will be described with respect to particular embodiments and with reference to certain drawings but the invention is not limited thereto. The described functions are not limited by the described structures. The drawings described are only schematic and are non-limiting. In the drawings, the size of some of the elements may be exaggerated and not drawn on scale for illustrative purposes.

Furthermore, the terms first, second, third and the like in the description and in the claims, are used for distinguishing between similar elements and not necessarily for describing a sequential or chronological order. The terms are interchangeable under appropriate circumstances and the embodiments of the invention can operate in other sequences than described or illustrated herein.

Furthermore, the various embodiments, although referred to as “preferred” are to be construed as exemplary manners in which the invention may be implemented rather than as limiting the scope of the invention.

The term “comprising”, used in the claims, should not be interpreted as being restricted to the elements or steps listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising A and B” should not be limited to devices consisting only of components A and B, rather with respect to the present invention, the only enumerated components of the device are A and B, and further the claim should be interpreted as including equivalents of those components.

On the figures, identical or analogous elements may be referred by a same number.

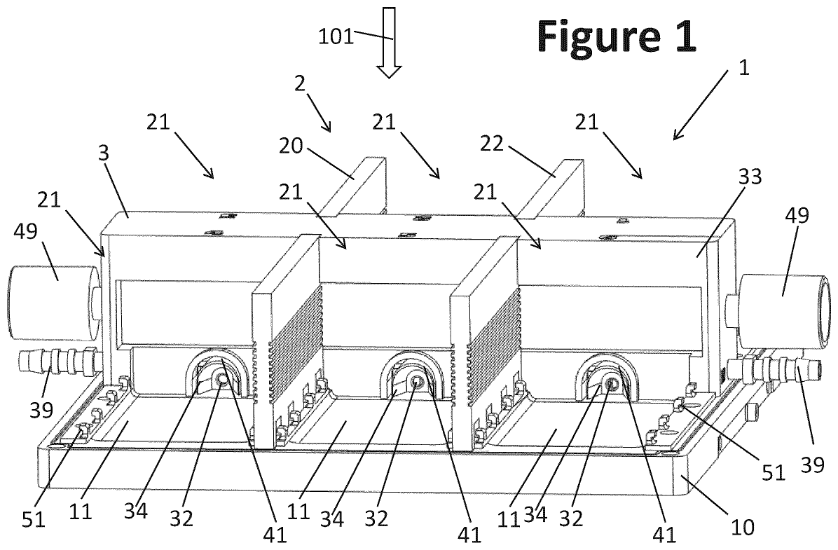

FIG. 1 illustrates some parts of a device 1 according to an embodiment of the invention. The device 1 is made to contain animals during their irradiation by a proton beam. The proton beam typically arrives on the device 1 parallel to the direction shown by the arrow 101 on FIG. 1. The device 1 comprises compartments 21 to receive animals. The device 1 is sealed in order to be as gastight as possible, and configured in such a way that the interior of each compartment 21 is reachable by the proton beam. The compartments 21 may, or not, be sealed from others.

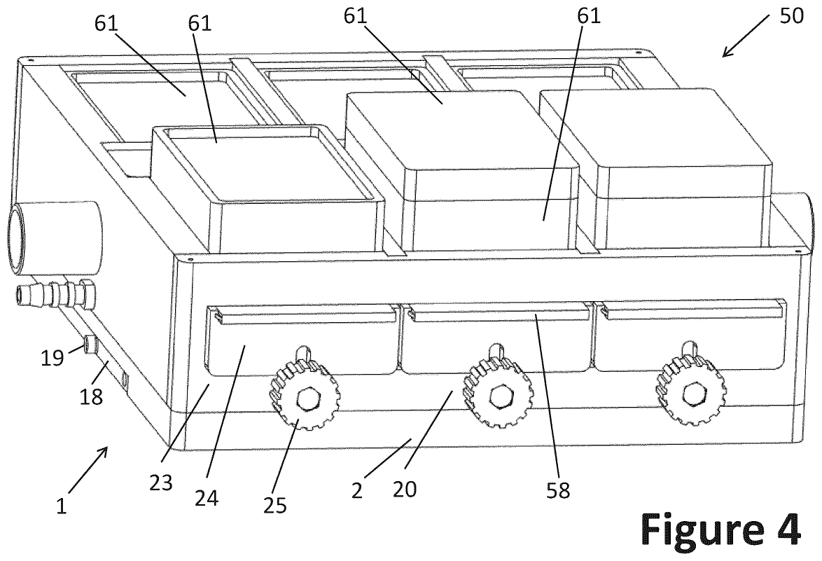

The device 1 comprises an enclosure 2 comprising a base plate 10, a lateral enclosure system 20, and a cover system 50 (FIG. 4). The base plate 10 supports the animal and closes a lower side of the enclosure 2. The lateral enclosure system 20 forms, at least partially, the separation between the compartments 21 and surrounds the enclosure 2 laterally to close it laterally. The cover system 50 closes a higher side of the enclosure 2. FIG. 1 shows only parts of the enclosure 2 and of the lateral enclosure system 20. The base plate 10, the lateral enclosure system 20, and the cover system 50 can be moved away from the others, for example to be cleaned separately.

The device 1 comprises a dispenser 3 for distributing an anesthetic gas to the compartments 21 in order to keep the animals asleep.

There is typically one animal per compartment 21 but there may be more than one animals or zero animal. The device 1 may comprise 6 compartments 21 as illustrated or more, or less. The compartments 21 may be arranged in two rows with one row on each side of the dispenser 3 or in any other way.

The device 1 may comprise hooks 51 on both sides of the compartments 21 in order to fasten straps maintaining the animals on the base plate 10. For example, the straps may be attached with a hook-and-loop (also known as Velcro) fixation or be, at least partly, in an elastic material. The device 1 may comprise fiducial markers, for example steal beads, in order to be placed correctly. The device 1 is preferably washed and sterilized between two uses.

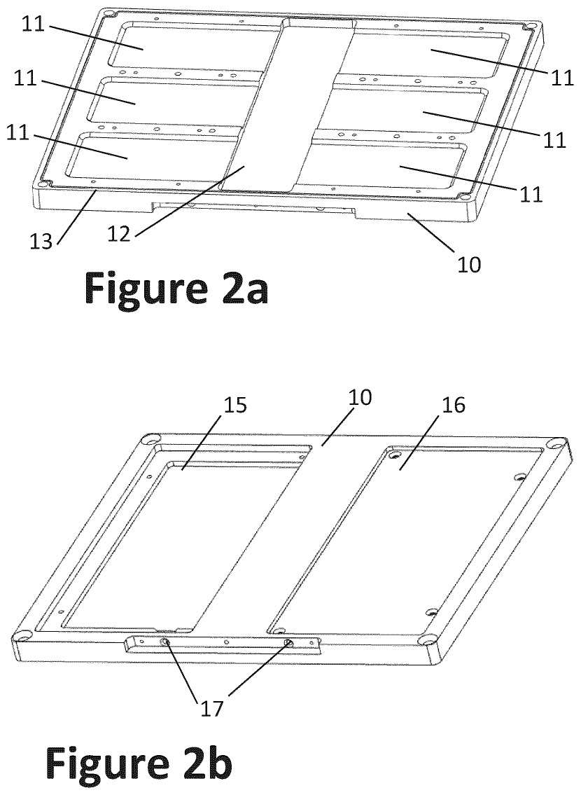

The base plate 10 will now be described referring mainly to FIGS. 1, 2a, and 2b. The base plate 10 preferably comprises a hollow 11 for each of the compartments 21, each hollow 11 being configured to support an animal and to collect fluids and outputs released by the supported animal. The base plate 10 may comprise a dispenser hollow 12 for supporting the dispenser 3. The base plate 10 may comprise a groove 13 surrounding the hollows 11 for receiving a gasket at the interface between the base plate 10 and the lateral enclosure system 20.

The base plate 10 forms a thermal contact between the animals and a heating arrangement. The heating arrangement may be at least partially located in one or several cavities 15 of the base plate 10. For example, the heating arrangement may comprise resistive heating pads located in the cavities 15. The cavities 15 are closed by plates 16 (at FIG. 2b, the plate 16 is only on the right cavity and the left cavity 15 is shown opened). The heating arrangement may be powered with cables passing through holes 17 opening in the inside of the cavities 15. In the embodiment illustrated FIG. 4, two through holes 17 are connected behind a junction piece 18 to a further hole 19 to group the cables. The heating arrangement may comprise a temperature sensor.

The lateral enclosure system 20 and the cover system 50 will now be described referring mainly to FIGS. 1, 3 and 4.

The lateral enclosure system 20 preferably comprises separation walls 22 forming at least some of the separations between the compartments 21, and surrounding wall 23. The surrounding wall 23 may comprise removable plates 24 fixed with screws 25.

The cover system 50 is preferably the part of the device 1 that receives the proton beam. The cover system 50 is preferably removable with respect to the base plate 10, and possibly to the lateral enclosure system 20. The cover system 50 preferably comprises a decelerating arrangement 60 and/or a shielding arrangement 52 configured in such a way that the proton beam crosses first the decelerating arrangement 60 and is then partially blocked by the shielding arrangement 52. The decelerating arrangement 60 may comprise parts that are fixed to the lateral enclosure system 20, and/or parts that are removable with respect to the lateral enclosure system.

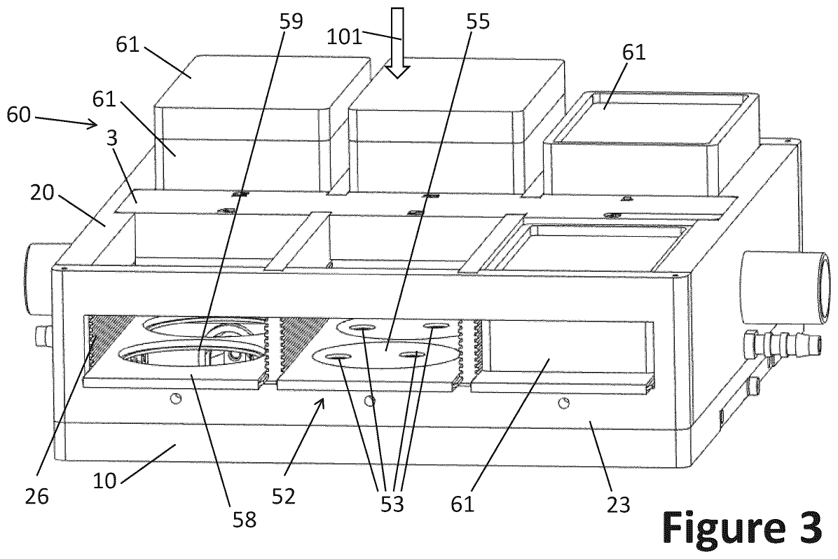

The decelerating arrangement 60 slows down the proton beam in order to decrease the energy of the proton beam. The decelerating arrangement 60 may comprise several decelerating elements 61 of various thicknesses. Each decelerating element 61 is configured for covering at least one of the compartments 21, and preferably not all compartments 21. In a preferred embodiment, each decelerating element 61 covers one of the compartments 21. At least some of the decelerating elements 61 may be configured to be stacked on each other. The decelerating elements 61 may have various thicknesses. In FIG. 3, in the front line of compartments 21, the decelerating arrangement 60 has been omitted on the two left compartments 21 and the right compartment 21 is covered by a decelerating element 61 of medium thickness. In the back line of compartments 21, the two left compartments 21 are covered by a decelerating element 61 of high thickness, which is itself covered by a decelerating element 61 of low thickness, and the right compartment 21 is covered only by a decelerating element 61 of high thickness.

The shielding arrangement 52 stops the proton beam except in some holes 53. The holes 53 are preferably parallel to the proton beam (arrow 101). The holes 53 are the only passages for the proton beam to the interior of the compartments 21. The device 1 may comprise plugs to close the holes 53 temporarily, for example in polyethylene. The device 1 may comprise proton beam collimators in the holes 53. The proton beam collimators may be made of two cylinders of different diameters, the narrower sliding inside the wider. They make possible to concentrate protons on a circle of, for example, 6 mm of diameter. The narrower cylinder may be placed in contact with the skin of the animals. They may be in tantalum, with a layer thickness of 3 mm.

The shielding arrangement 52 may comprise a shielding element for each of the compartments. The lateral enclosure system 20 may comprise a system of height adjustment 26 for placing the shielding elements at different heights. The height can thus be adapted to the format of the animal.

The shielding arrangement 52 may comprise a movable arrangement movable with respect to the dispenser 3, and thus with respect to the base plate 10 and the animals. The holes 3 are preferably in the movable arrangement in order to move the part of the proton beam received by the animals when the arrangement movable is moved.

In the exemplary embodiment of FIG. 3, the movable arrangement comprises, for each compartment 21, a shielding element comprising a plate 58 with a through hole 59, and a movable element 55 in the through hole 59. The plate 58 can be shifted towards or away from the dispenser 3, and the movable element 55 is rotatable around an axis parallel to the holes 53, i.e. preferably around the direction of the proton beam (arrow 101). For example, the tumors to irradiate may be located in the paws and the abdomen of the animals. In order to reach each of the four paws one after each other, the plate 58 is shifted towards or away from the dispenser 3 and the movable element 55 is rotated. If a part of the compartment 21 is not covered by the shielding element because of the translation of the plate 58, it is not scanned by the proton beam.

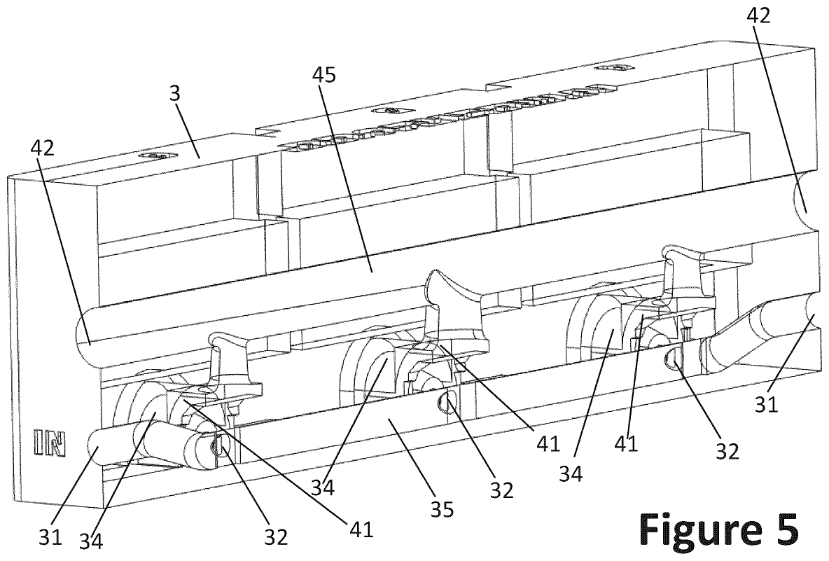

The dispenser 3 will now be described referring mainly to FIGS. 1, and 5. The dispenser 3 is surrounded, laterally, by a lateral wall 33. The dispenser 3 comprises at least one gas inlet 31, which opens outside the devices 1. The gas inlet 31 is preferably connected to a fluid connection element 39, which is connected to an anesthetic container.

The dispenser 3 comprises several gas outlets 32, which open in the compartments 21 and are connected to the gas inlet 31 by a gas line 35. Each compartment 21 may have one or several gas outlets 32. The gas outlets 32 are preferably in the lateral wall 33. They may be in recesses 34 of the lateral wall 33, each recess 34 being configured to receive the mouse and/or nose of the animal.

In an embodiment of the invention, the device 1 comprises a suction system for the anesthetic gas. The suction system is preferably located in the dispenser 3. The suction system comprises at least one gas outlet 42, which opens outside the devices 1. The gas outlet 42 is connected to a fluid connection element 49, which is connected to a suction pump.

The suction system comprises several suction inlets 41, which open in the compartments 21 and are connected to the gas outlet 42 by a suction line 45. The suction line 45 is preferably parallel to the gas line 35.

Each compartment 21 may have one or several suction inlets 41. The suction inlets 41 are preferably in the lateral wall 33. They may be in the recesses 34 of the lateral wall 33. For example, as illustrated on FIGS. 1 and 5, each gas outlet 32 may be partially surrounded by a suction inlet 41.

The device 1 is preferably transportable with a transport arrangement. An equipment according to the invention includes a device according to the invention and a transport arrangement. In an embodiment of the invention, the transport arrangement is a trolley supporting the device 1 and at least one of: the anesthetic container, an oxygen bottle connected to the gas inlet 31 to diffuse the anesthetic gas from the container to the dispenser 3, the suction pump, and a battery for powering the elements of the equipment. The trolley may also support a chamber for putting the animals to sleep. This chamber is only a backup because the animals are generally put to sleep in another room than the room where the proton beam arrives.

In other words, the invention relates to a device 1 for containing animals and keeping them asleep during their irradiation by a proton beam. The animals are located in compartments 21 and kept asleep with an anesthetic gas provided to them by a dispenser 3. The device 1 is sealed, which prevents escape from the animals and leakage of anesthetic gas.

Although the present invention has been described above with respect to particular embodiments, it will readily be appreciated that other embodiments are also possible.

1. A device for containing animals during irradiation by a proton beam, comprising:
an enclosure delimiting, at least partially, several compartments, each compartment comprising an interior suitable for receiving an animal; and
a dispenser configured for dispensing an anesthetic gas into the compartments,
wherein the device is sealed and configured in such a way that the interior of each compartment is reachable by the proton beam;
wherein the enclosure comprises a cover system;
wherein the cover system comprises a decelerating arrangement configured to decrease the energy of the proton beam, the energy being adapted according to the depth of the tissue to irradiate.

2. The device according to claim 1, wherein the enclosure comprises a base plate configured to support the animals and to close a lower side of the enclosure, and a lateral enclosure system configured to create a partition between the compartments and to close the enclosure laterally.

3. The device according to claim 2, wherein the base plate comprises a hollow for each of the compartments, each hollow being configured to support an animal and to collects fluids and outputs released by the supported animal.

4. The device according to claim 2, comprising a heating arrangement in thermal contact with the supported animals through the base plate.

5. The device according to claim 1, wherein cover system is configured to close a higher side of the enclosure.

6. The device according to claim 1, wherein the cover system is, at least partially, movable.

7. The device according to claim 1, wherein the decelerating arrangement comprises several decelerating elements of various thicknesses, configured in such a way that at least two of the compartments are covered by decelerating elements of different thicknesses.

8. The device according to claim 1, wherein the cover system comprises a shielding arrangement blocking the proton beam except in several holes.

9. The device according to claim 8, comprising proton beam collimators in the holes.

10. The device according to claim 8, wherein the holes are in a movable arrangement of the shielding arrangement, the movable arrangement being movable with respect to the dispenser in such a way that a motion of the movable arrangement moves the holes with respect to the animals.

11. The device according to claim 10, wherein the holes are in movable elements of the movable arrangement, the movable elements being rotatable around an axis parallel to the holes.

12. The device according to claim 1, wherein the dispenser comprises a gas inlet and several gas outlets, each gas outlet opening into one of the compartments.

13. The device according claim 12, wherein the gas outlets are in a lateral wall of the dispenser, preferably in recesses of the lateral wall of the dispenser.

14. The device according to claim 1, further comprising a suction system for the anesthetic gas, the suction system comprising several suction inlets and a suction outlet, each suction inlet opening into one of the compartments.

15. Equipment comprising a device for containing animals during irradiation by a proton beam, the device comprising:
an enclosure delimiting, at least partially, several compartments, each compartment comprising an interior suitable for receiving an animal; and
a dispenser configured for dispensing an anesthetic gas into the compartments,
wherein the device is sealed and configured in such a way that the interior of each compartment is reachable by the proton beam;
wherein the enclosure comprises a cover system;
the cover system comprises a decelerating arrangement configured to decrease the energy of the proton beam, the energy being adapted according to the depth of the tissue to irradiate, and
a transport arrangement configured to move the device.
